(19) United States
(12) Patent Application Publication
(10) Pub. No.: US 2006/0166905 A1
(43) Pub. Date: Jul. 27, 2006

(54) Aivlosin for the treatment of disease due to brachyspira pilosicoli or ornithobacterium rhinotracheale

(75) Inventor: Michael Sanders, London (GB)

Correspondence Address: VENABLE LLP, P.O. BOX 34385, WASHINGTON, DC, 20045-9998, US

(73) Assignee: ECO Animal Health, LTD., London (GB)

(21) Appl. No.: 10/563,210

(22) PCT Filed: Jul. 4, 2004
(86) PCT No.: PCT/GB04/02887

§ 371 Date: Jan. 3, 2006

(30) Foreign Application Priority Data
Jul. 3, 2003 (GB) 0315629.6

(51) Int. Cl. A61K 31/7048

(52) U.S. Cl. 514/28

(57) ABSTRACT

The invention relates to the use of Aivlosin for the treatment, prevention or control of diseases due to Brachyspira pilosicoli in pigs and Ornithobacterium rhinotrachelae in poultry.

The present invention relates to the use of antibiotics as medicaments for the treatment or prevention and control of disease and infections especially in pigs and poultry. Pigs and poultry, especially those which are intensively reared or reared in large-scale operations, have a tendency to suffer from or risk catching a variety of diseases and infections, for example, diseases due to Brachyspira pilosicoli in pigs and Ornithobacterium rhinotracheale in poultry.

Brachyspira pilosicoli causes a condition known as spirochetal diarrhoea, which features diarrhoea and slow growth. The infection rarely causes death, but can be fatal, particularly when associated with infection with other organisms, for example another intestinal parasite. Infection can have a major economical impact on the profitability of pig production due to reductions in daily weight gain and feed conversion. Unlike some other porcine intestinal infections, disease due to Brachyspira pilosicoli is not solely associated with pigs. It is also known to infect humans, dogs and other animals. Ornithobacterium rhinotracheale is a respiratory disease characterised by mild respiratory signs, increased mortality, poor weight gain and feed conversion, brain lesions, and reduced egg production. It causes economic losses due to increased medical costs, reduced growth and high condemnation rates at processing. Ornithobacterium rhinotracheale has been isolated from many species including chicken, duck, partridge, goose, pigeon, and turkey, amongst others. This indicates there is a broad potential reservoir.

Although there are some known treatments for Brachyspira pilosicoli and Ornithobacterium rhinotracheale, they are frequently ineffective due to widespread resistance to commonly used antibiotics.

Surprisingly, the inventors have found that the known antibiotic aivlosin (otherwise known as 3-O-acetyl-4″-O-isovaleryl-tylosin), which has previously been used in high doses for the treatment and control of Mycoplasma diseases in poultry, is also effective in the prevention or control of Brachyspira pilosicoli, particularly in pigs and of Ornithobacterium rhinotracheale particularly in poultry.

In British Patent Specification No. 1,539,907 there are disclosed tylosin derivatives having acyl groups in the 3 and 4″ positions and acid addition salts thereof, specifically the tartaric, acetic, propionic, citric, succinic, hydrochloric, sulphuric and phosphoric acid addition salts. Amongst the tylosin derivatives specifically disclosed there is 3-O acetyl-4″-O-isovaleryl-tylosin, which is now commonly known as aivlosin. This compound has the formula 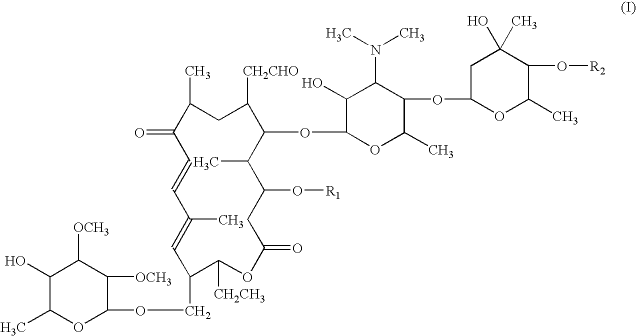
where R₁ is acetyl and R₂ is isovaleryl. There is also disclosed a process for the production of aivlosin by the biochemical acylation of tylosin or an appropriately partially acylated tylosin by means of an appropriate acylating micro-organism of the genus Streptomyces, especially one selected from Streptomyces thermotolerans (ATCC 11416), Streptomyces fungicidus subsp. espinomyceticus (ATCC 21574), Streptomyces mycarofaciens (ATCC 21454) and Streptomyces hygroscopicus (ATCC 21582), in the presence of the appropriate acyl donor, especially acetyl CoA, isovaleryl CoA, acetic acid, isovaleric acid, potassium, sodium or ammonium salts of those acids, methanol and ethanol esters of these acids, amides of these acids and α-oxovaleric acid.

British Patent Specification No. 1,539,907 mentions that the tylosin derivatives can be administered to humans or animals and refers to their activity against a number of gram-positive bacteria, including some drug-resistant bacteria, but it does not specifically refer to the use of the derivatives in the treatment or control of specific diseases or infections of animals, although it does say that they can be employed on humans, livestock, household pets, laboratory animals and poultry and in the enteral, parenteral or topical control of infectious diseases in a similar manner as for known macrolide antibiotic drugs.

In fact, aivlosin on the basis of its initial Japanese marketing registration (No 4 chika AC1771) has to date been marketed and approved for marketing only for the treatment and control of Mycoplasma diseases in pigs and poultry at high doses of 200 to 500 ppm in feed. There should be no reason to suppose that it would be suitable for the treatment and prevention and control of other infections and diseases, particularly of pigs and poultry. Other macrolide antibiotics, such as erythromycin, having an effectiveness against mycoplasma diseases, do not have any effect or any significant effect against other infections of pigs and poultry such as diseases due to Brachyspira pilosicoli and Ornithobacterium rhinotracheale. It is, of course, a feature of the approvals schemes which apply in all major countries that a veterinary medicament which is approved for marketing for one specific purpose cannot be marketed or recommended for use for any other specific purpose without a separate authorisation or approval from the relevant authority. There is thus a strong counter-incentive to the research into the use of even known antibiotics for new veterinary uses.

PCT application WO 02/32233 describes the use of aivlosin to treat Brachyspira hyodysenteriae in pigs. Brachyspira hyodysenteriae is an infection of the large intestine. It causes swine dysentery, resulting in bloody diarrhoea and death through dehydration. The activity of aivlosin against disease due to Brachyspira pilosicoli could not have been predicted from the use against Brachyspira hyodyseteriae because the efficacy of any antimicrobials cannot be accurately defined without tests on live animals.

WO 02/32233 also discloses the use of aivlosin to treat Lawsonia intracellularis in pigs. Lawsonia intracellularis is a disease of the small intestine which causes diarrhoea and wasting. Lawsonia intracellularis can be categorised as a Gram negative organism, but it is not a conventional Gram negative bacterium.

Aivlosin is characterised as a macrolide antibiotic, effective against Gram positive bacteria and mycoplasma. Such antibiotics are not expected to be effective against Gram negative bacteria (Antimicrobial Therapy in Veterinary Medicine, 3rd Ed. (2000) Edited by Prescott J F, Baggot J D and Walker R D. Iowa State University Press), as demonstrated by existing data of the use of macrolides against Pasteurella haemolytica. Activity of macrolides cannot be predicted from the bacterial wall structure. Macrolides have been demonstrated to be effective against both Mycobacterium spp., the cell walls of which incorporate complex lipid conjugates and the wall-less Mycoplasma spp. The activity of any macrolide cannot be predicted without specific testing. Against expectation, the inventors have found that it is also effective against the Gram negative bacteria Ornithobacterium rhinotracheale.

From extensive in vitro and in vivo (animal) trial work, the inventors have confirmed that aivlosin and acceptable derivatives thereof are effective in the prevention and control and treatment of disease due to Brachyspira pilosicoli in pigs and Ornithobacterium rhinotracheale in poultry at reasonable dose rates.

The present invention provides for the use of aivlosin, as such or as a pharmacologically acceptable (non-toxic) derivative, such as an acid addition salt, in the preparation of a medicament for the treatment or prophylaxis of disease due to Brachyspira pilosicoli and Ornithobacterium rhinotracheale in animals. The medicament is preferably for treating disease due to Brachyspira pilosicoli in mammals, more preferably in pigs. In an alternative embodiment the medicament is also for treating Ornithobacterium rhinotracheale in birds, more preferably in poultry. Also provided is a method of treatment or control of disease due to Brachyspira pilosicoli and Ornithobacterium rhinotracheale in animals comprising administering to an animal an effective amount of aivlosin or a pharmacologically effective derivative thereof.

The term prophylaxis means prevention or control of a disease. It encompasses both stopping disease occurring, and keeping disease at a manageable level.

The term “disease due to” means a disruption of an animal's physiological state because of infection by Brachyspira pilosicoli. The disruption in physiological state includes specific symptoms or signs that are related to Brachyspira pilosicoli infection, and simply an overall reduction in health, which for example may result in the animal being more susceptible to infection or disease from other pathogens. In particular disease due to Brachyspira pilosicoli includes spirochetal diarrhoea.

The term “pigs” encompasses all members of the pig family, for example, members of the Suidae family. The term “poultry” encompasses all types of domestic fowl, including, but not limited to chickens, turkey, ducks, geese, the ratite group of birds and game birds.

Preferably the medicament for the treatment or prevention or control of disease due to Brachyspira pilosicoli in pigs is added to food at a level of 10 to 200 ppm, more preferably 10 to 100 ppm, even more preferably 20 to 50 ppm.

Preferably the medicament for the treatment or prevention or control of Ornithobacterium rhinotracheale in poultry is added to food at a level of 10 to 200 ppm, more preferably 10 to 100 ppm, even more preferably 20 to 50 ppm. The medicament for the treatment or prevention and control of Ornithobacterium rhinotracheale in poultry can also be added to water in water soluble form at a dose of 10 to 100 mg per 1 kg body weight, and more preferably 20 to 40 mg per 1 kg.

Either medicament is preferably suitable for addition to food or drinking water. Alternatively the medicament may be suitable for administration by injection.

Also provided by the invention is the use of aivlosin to prevent or reduce growth of Brachyspira pilosicoli or Ornithobacterium rhinotracheale in vitro. The prevention or reduction of growth of these bacteria can be useful in the in vitro preparation of intestinal tissues, or in the comparison of the activity of antibiotics against bacteria.

Aivlosin is available in free form as a white crystalline granule having a melting point of 180° C.-184° C., soluble in lower alcohols such as ethanol, ketones such as acetone, ethers such as diethyl ether, esters such as ethyl acetate and aromatic hydrocarbons such as toluene, although it is barely soluble in n-hexane and petroleum ether. It is very soluble in aqueous solutions of pH around and below 7 but less soluble in aqueous solutions of higher pH. Because it is a basic compound it forms acid addition salts, and the use of such salts which are pharmacologically acceptable is also included within the present invention. Acids to form acceptable acid addition salts include inorganic acids such as hydrochloric, sulphuric or phosphoric acid and organic acids such as tartaric, acetic, propionic, citric and succinic acids. Specific examples of acceptable derivatives are aivlosin hydrochloride (melting point 129-133° C.) and aivlosin tartrate (melting point 119-122° C.). Such derivatives are frequently more water-soluble than aivlosin itself and their use may therefore have formulation advantages.

Derivatives of aivlosin preferably include any pharmacologically acceptable functional derivatives. The functional derivatives may be produced by modifying one or more of the substituent groups of aivlosin. Preferably the derivative is a salt; most preferably an acid salt.

Aivlosin and appropriate derivatives can be formulated according to the present invention into medicaments in known ways, for example to provide compositions for oral, enteral or parenteral administration, by admixing with appropriate solid or liquid carriers and excipients for the administration route desired. Conventional ingredients can be used as carriers and excipients, for example water and salt solutions for liquid formulations and silicaceous materials-silica and silicates (such as hydrated magnesium silicate), cereal products (such as soybean meal and wheat flour) and other pharmacologically acceptable solids for solid formulations for oral administration. The formulations can also contain further auxiliaries and additives such as minerals, lubricants, preservatives, stabilisers, wetting agents, emulsifiers, buffers and colouring or flavouring materials in a conventional manner. In the prophylaxis or control of the diseases mentioned it is particularly convenient to include the aivlosin or derivative as an additive to animal feed or drinking water for animals, but in the treatment of the disease it can be included in an injectable solution, or a tablet, capsule or syrup, if desired.

Aivlosin (as such or in the form of an appropriate derivative, for example an acid addition salt such as the tartrate) may be formulated into premixes in various potencies from 1 to 10% by weight. A particularly suitable composition for producing such premixes comprises aivlosin salt, filler such as soybean powder and additives such as hydroxypropyl cellulose and has a potency of 180 to 220 mg/g.

In order to ensure stability of aivlosin in animal feed which may have been subjected to high-temperature processing for pelleted or extruded feed it is desirable to provide a coated aivlosin (as such or in the form of an appropriate derivative, for example an acid addition salt such as the tartrate) in particulate form coated with polyvinylpyrrolidone. Suitable proportions by weight are in the range of 50:1 to 1:1 active ingredient: polyvinylpyrrolidone. Inert fillers and other ingredients may be present in such compositions, the overall polyvinylpyrrolidone concentration being preferably 0.1 to 10% by weight.

The medicament formulations for use either as feed additives or as directly administered preparations may contain any convenient proportion of aivlosin for example from 1% or less to 90% or more, by weight. Liquid formulations typically contain 50 to 90% by weight, whereas solid formulations typically contain 1 to 25% by weight.

For treatment or prevention or control of diseases in pigs due to Brachyspira pilosicoli aivlosin may for example be administered in feed at a rate of 10 to 200 ppm by weight (10 to 200 g per 1,000 kg of feed) for a period of time long enough to control or treat the disease successfully, for example 7-14 days.

For treatment or prevention or control of Ornithobacterium rhinotracheale infections in poultry aivlosin may for example be administered in feed at a rate of 20 to 50 ppm by weight (20 to 50g per 1,000 kg of feed) for a period of time long enough to control or treat the disease successfully, for example 7-14 days. Alternatively aivlosin may be administered at a rate of between 100 to 250 ppm by weight in drinking water (100 to 250g) per 1000 l of water), preferably between 100 and 150 ppm by weight.

The following examples, in which parts are by weight, illustrate the use of aivlosin in the manufacture of veterinary medicaments or preparations for treatment or prophylaxis of the animal infections according to the present invention.

### EXAMPLE 1

20 parts of aivlosin API (active pharmaceutical ingredient) made into a solution in water is mixed with 80 parts of soybean meal, and the mixture is spray dried to give a solid additive for feedstuff containing 200 kg aivlosin activity per 1000 kg. This formulation can be added to pig and poultry feed to provide an in-feed concentration of aivlosin of 25 to 200 g aivlosin per 1000 kg final feed.

### EXAMPLE 2

25 parts of aivlosin 20% is mixed with 50 parts of hydrated magnesium silicate (an inert silica), 24 parts of wheat feed flour and 1 part of liquid paraffin EP as a powder blend to give a solid additive for feedstuff containing 50 kg aivlosin activity per 1000 kg. This formulation can be used in pig and poultry feed as in Example 1.

### EXAMPLE 3

5 parts of aivlosin 20% as used in Example 2 is mixed with 40 parts of hydrated magnesium silicate, 54 parts of wheat feed flour and 1 part of liquid paraffin EP as a powder blend to give a solid additive for feedstuff containing 10 kg aivlosin activity per 1000 kg. This formulation can be used in pig and poultry feed as in Example 1.

### EXAMPLE 4

Aivlosin is dissolved in water to provide an aqueous solution containing 80-90% aivlosin activity for use in drinking water for pigs or poultry. This formulation can be added to drinking water to provide aivlosin concentrations in drinking water in the range 25 to 100 g per 200 litres of drinking water.

### EXAMPLE 5

Aivlosin API containing more than 80% w/w aivlosin tartrate was mixed into an 850 kg batch comprising

| | | |
|---|---|---|
| Aivlosin API | 163-169 | kg |
| Hydroxypropyl cellulose. Ph. Eur. | 8.2-8.5 | kg |
| Water, Ph. Eur | 800-1200 | litres |
| Non-fat soybean powder | 720 | kg |

The batch was processed and the water was removed during processing. The input of aivlosin API was adjusted for content value of free base, determined by HPLC, of the raw material to achieve a final product bioassay potency of 180-220 mg/g. The product (AIVLOSIN FG 200), which could also be produced in other batch sizes, was suitable for manufacturing aivlosin premixes in various potencies from 1% to 10%.

### EXAMPLE 6

Coated aivlosin formulations possessing stability in animal feed after high-temperature processing for pelleted or extruded feed were produced in batches of 1000 kg (although other batch sizes could be used) from the following ingredients:

| | | |
|---|---|---|
| AIVLOS1N FG 200 (see Example 5) | 250.0 | kg |
| Paraffin, Light Liquid, Ph. Eur. | 10.0 | kg |
| Wheat feed flour | 240.0 | kg |
| Polyvinylpyrrolidone | 10.0 kg-100.0 | kg |
| Sepiolite | to 1000.0 | kg |

### EXAMPLE 7

### Determination of Minimum Inhibitory Concentration (MIC) of Aivlosin Against Brachyspira pilosicoli

The MIC of acetyl isovaleryltylosin was determined against 5 field strains of Brachyspira pilosicoli isolated from different pig populations in various parts of England using an antibiotic dilution method. Four replicates of 0.2 ml of each isolate were inoculated into agar plates prepared with antibiotic concentrations from 0.78 to 200 μg/ml. A strain of Brachyspira hyodysenteriae (P18A), for which the MIC for acetyl isovaleryltylosin was already known, was used as a control. The determined MICs are given in the table below.

| Strain Ref. | MIC (μg/ml) |
|---|---|
| P0098-10-97 | 25.0 |
| P0352-10-98 | 6.25 |
| P0730-09-97 | 12.5 |
| 0P0204-10-97(4) | <0.781 |
| P0525-01-98 | 6.25 |
| Control P1 8A | 12.5 |

It is concluded that the MICs for acetyl isovaleryltylosin were generally similar, falling in the range 6.25-25.0 μg/ml with the exception of one isolate (P0204-10-97 (4)) against which acetyl isovaleryltylosin was far more active.

The results show that aivlosin is particularly effective at preventing the growth of Brachyspira pilosicoli, even at relatively low concentrations.

### EXAMPLE 8

### Testing of Aivlosin and Tilmicosin (Pulmotil) Against Ornithobacterium rhinotracheale Using the MIC (Minimum Inhibitory Concentration) Test

The following antibiotics were each tested against four isolates of Ornithobacterium rhinotracheale (OR):

1) Aivlosin

2) Pulmotil

The antibiotics were tested against the following isolates of OR:

1) 568/99

2) 587/00

3) 33/01

4) 1322/01

1) MIC Method

The concentration needed of each antibiotic was calculated according to its active ingredient.

Aivlosin

81% active

32 μg/ml×2=64 μg/ml

64 μg/ml×1.23=78.72 μg/ml

=0.04 g/500 ml

Pulmotil

25% active

32 μl/ml×2=64 μl/ml

64 μl/ml×4=256 μl/ml

=0.13 ml/500 ml

The four freeze dried bacterial isolates of Ornithobacterium rhinotracheale were reconstituted, inoculated into 10 ml serum broth (supplied by OBP, code 655) and incubated at 37° C. for 48 hours. After incubation the optical density (OD) of each culture was read at 540 nm. To verify purity the cultures were plated out onto Blood Tryptose agar plates, incubated at 37° C., using 5 % CO₂ conditions, for 48 hours after which the plates were examined for any contaminants.

12 tubes per isolate for each of the antibiotics were placed into a test tube rack. 2 ml serum broth was dispensed into each of the tubes. 2 ml of the antibiotic were then added to the first tube in the respective row and a twofold dilution were made (32 μg/ml-0.0625 μg/ml). 20 μl of the bacteria was then added to 11 of the tubes. The 12^{th} tube, i.e. negative control received neither antibiotic nor bacteria. The 11^{th} tube i.e. positive control only received 20 μl bacteria. All the tubes were then incubated at 37° C. and the MIC read after 48 hours incubation

2) Results

Optical density readings (540 nm) before addition to antibiotic dilutions:

1) 568/99-1.025

2) 587/00-1.045

3) 33/01-1.058

4) 1322/01-1.081

1) Aivlosin

| Dilution | Bacterial Growth | | | |
|---|---|---|---|---|
| (μg/ml | 568/99 | 587/00 | 33/01 | 1322/01 |
| 32 | − | − | − | − |
| 16 | − | − | − | − |
| 8 | − | − | − | − |
| 4 | − | − | − | − |
| 2 | − | − | − | − |
| 1 | − | − | − | − |
| 0.5 | − | − | − | − |
| 0.25 | + | + | + | + |
| 0.125 | + | + | + | + |
| 0.0625 | + | + | + | + |
| Negative control | − | − | − | − |
| Positive control | + | + | + | + |

2) Pulmotil

| Dilution | Bacterial Growth | | | |
|---|---|---|---|---|
| (μg/ml | 568/99 | 587/00 | 33/01 | 1322/01 |
| 32 | − | − | − | − |
| 16 | − | − | − | − |
| 8 | − | − | − | − |
| 4 | − | − | − | + |
| 2 | + | + | + | + |
| 1 | + | + | + | + |
| 0.5 | + | + | + | + |
| 0.25 | + | + | + | + |
| 0.125 | + | + | + | + |
| 0.0625 | + | + | + | + |
| Negative control | − | − | − | − |
| Positive control | + | + | + | + |

13) Conclusions:

A concentration of 0.5 μg/ml Aivlosin inhibited the growth of the isolates of Ornithobacterium rhinotracheale tested.

A concentration of 4 μg/ml Pulmotil inhibited the growth of three of the isolates of Ornithobacterium rhinotracheale tested Inhibition of the fourth isolate was only achieved at a concentration of 8 μg/ml.

The results show that aivlosin is particularly effective at preventing the growth of Ornithobacterium rhinotracheale, even at relatively low concentrations.

1. The use of aivlosin or a pharmacologically acceptable derivative thereof for the preparation of a medicament for the treatment or prevention or control of disease due to Brachyspira pilosicoli.

2. The use of claim 1 wherein the medicament is for treating pigs.

3. The use of claim 1 wherein the medicament is for addition to feed at a rate of 10 to 200 ppm.

4. The use of claim 1 wherein the medicament is an additive for feed or drinking water.

5. A method of treating or preventing or controlling disease due to Brachyspira pilosicoli comprising administering to an animal an effective amount of aivlosin or a pharmacologically acceptable salt thereof.

6. The method of claim 5 wherein the animal is a pig.

7. The method of claim 5 wherein the aivlosin or a pharmacologically acceptable derivative thereof is administered in either feed or drinking water.

8. The use of aivlosin or a pharmacologically acceptable derivative thereof for the preparation of a medicament for the treatment or prevention or control of an Ornithobacterium rhinotracheale infection.

9. The use of claim 8 wherein the medicament is for treating poultry.

10. The use of claim 8 wherein the medicament is for addition to feed at a rate of 10 to 200 ppm.

11. The use of claim 8 wherein the medicament is an additive for feed or drinking water.

12. A method of treating or preventing or controlling an Ornithobacterium rhinotracheale infection in animals comprising administering to an animal an effective amount of aivlosin or a pharmacologically acceptable derivative thereof.

13. The method of claim 12 wherein the animal is a poultry bird.

14. The method of claim 13 wherein the aivlosin or a pharmacologically acceptable derivative thereof is administered in either feed or drinking water.

15. The use of aivlosin or a pharmacologically acceptable derivative thereof in the prevention or reduction of growth of Brachyspira pilosicoli in vitro.

16. The use of aivlosin or a pharmacologically acceptable derivative thereof in the prevention or reduction of growth of Ornithobacterium rhinotracheale in vitro.
